(19) United States
(12) Patent Application Publication
(10) Pub. No.: US 2013/0345143 A1
(43) Pub. Date: Dec. 26, 2013

(54) MODULATION OF p62 FUNCTION THROUGH THE PB1 DOMAIN

(71) Applicant: Rutgers, The State University of New Jersey, (US)

(72) Inventors: Eileen White, Princeton, NJ (US); Jessie Yanxiang Guo, Belle Mead, NJ (US); Gizem Karsli Uzunbas, Piscataway, NJ (US)

(73) Assignee: RUTGERS, THE STATE UNIVERSITY OF NEW JERSEY, New Brunswick, NJ (US)

(21) Appl. No.: 13/755,562

(22) Filed: Jan. 31, 2013

Related U.S. Application Data
(60) Provisional application No. 61/593,068, filed on Jan. 31, 2012.

(51) Int. Cl. C07K 14/435

(52) U.S. Cl. CPC C07K 14/435
USPC 514/19.3; 530/300; 514/21.3; 536/23.5; 435/375; 435/325; 800/13

(57) ABSTRACT

Provided are methods, compositions, and kits employing a molecule that exhibits a function of the PB1 domain. In particular, such methods, compositions, and kits may be used for inhibiting tumorigenesis. Also provided are molecules that exhibit a function of the PB1 domain and methods for making such molecules. Additionally provided are cell lines that express a polypeptide having a function of the PB1 domain, as well as transgenic animals that express a polypeptide having a function of the PB1 domain.

### CROSS-REFERENCE TO RELATED APPLICATIONS

This application claims the benefit of U.S. Provisional Application No. 61/593,068, filed Jan. 31, 2012, the disclosure of which is hereby incorporated by reference herein in its entirety.

### STATEMENT REGARDING FEDERALLY SPONSORED RESEARCH

This invention was made, at least in part, with government support under grant number NIH R01 CA130893 awarded by the National Institutes of Health. The U.S. government has certain rights in the invention.

### BACKGROUND OF THE INVENTION

The balance between synthesis and breakdown of proteins maintains cellular homeostasis. In eukaryotic cells proteins can be degraded through two independent mechanisms. Short-lived proteins are degraded through ubiquitin-proteosome system, whereas long-lived and damaged proteins/organelles are degraded through a highly regulated catabolic self-degradation process called autophagy (Noda, et al., 2009, Chemical reviews, 109(4):1587-1598), which maintains metabolic homeostasis through recycling, of cellular components such as damaged organelles and proteins for its biomass (Mizushima and Klionsky, 2007, Anna Rev Nutr, 27:19-40).

Autophagy functions to sequester cargo, such as damaged organelles and proteins, in a double membrane vesicle called autophagosome (Mizushima, et al., 2008, Nature, 451(7182): 1069-1075). The cytosolic material is first sequestered by an expanding membrane sac called phagophore; then by expansion of phagophore, the double membrane vesicle autophagosome is formed. Finally, these vesicles fuse with lysosome to form the autolysosome and contents are degraded by lysosomal acidic hydrolases. Macromolecules are then released to the cytosol by permeases for recycling (Xie and Klionsky, 2007, Nat Cell Biol, 9(10): 1102-1109)

Autophagy is activated in response to extra- and intro-cellular stress, such as starvation, growth factor deprivation and infection and prevents toxic accumulation of unfolded and easy-to-aggregate proteins, lipids and damaged organelles. mTOR, a serine/threonine kinase regulating cell growth and survival, is a key sensor molecule involved in autophagy regulation (Cecconi and Levine, 2008, Dev Cell, 15(3): 344-357). In response to nutrient levels, mTOR can trigger protein translation through phosphorylation of several proteins that leads to increased synthesis of proteins required for protein synthetic machinery. In nutrient deprivation, mTOR repression moves cellular metabolism toward autophagy, which recycles the components required for survival.

Initiation of autophagy occurs through activation of autophagy related genes (ATG). Autophagosome formation is the first step of autophagy, and Ulk1 (the serine/threonine kinase) and Beclin-1 (the myosin-like Bcl-2 interacting protein) are early genes involved in autophagosome generation (Tomoda, et al., 1999, Neuron, 24(4): 833-846). There are two ubiquitin-like conjugation systems involved in the expansion and closure of the autophagosome. Similar to ubiquitin, Atg7 behaves as an E1-like ubiquitin activating enzyme and Atg10 behaves as an E2-like conjugating enzyme to transfer the small ubiquitin-like Atg12 protein to Atg5 (Mizushima, et al, 1998, Nature, 395(6700): 395-398). Atg5-Atg12 then associates with Atg16L, (Atg16-like) to form a multimeric complex This covalent linkage is essential for the modification of LC3, where the lipid attachment occurs. In that second conjugation reaction, LC3 (microtubule-associate protein 1 light chain-3), another ubiquitin-like molecule, is processed by the cysteine protease Atg4 to expose a C-terminal glycine; this form is called LC3-I. Cytosolic LC3-I is activated by cleavage and linkage to phosphatidylethanolamine (PE) by E1-like Atg7 and E2-like Atg3 and then translocates from cytoplasm to autophagosomal membrane where lipid attaches to LC3-I to make membrane bound LC3-II (FIG. 1) (Ichimura, et al., 2000: Nature, 408(6811): 488-492; Cecconi, F. and B. Levine (2008). Developmental Cell 15(3): 344-357). The mature autophagosome, bearing the cargo inside, fuses to the lysosome, giving rise to a structure called the autolysosome in which the acidic proteases digest the cargo to recycle amino acids, carbohydrates, fatty acids and nucleotides to be utilized under stress conditions.

p62 (Sequestosome1; SQSTM1) is an important protein in the autophagy machinery due to its role in trafficking proteins to the autophagosome (FIG. 2) (Chen and White, 2011, Cancer Prevention Research 4(7): 973-983; Komatsu and Ichimura, 2010, FEBS Letters 584(7): 1374-1378). p62/Sqstm1 is an autophagy scaffold protein responsible for trafficking damaged mitochondria and unfolded proteins to the lysosome. p62 binds ubiquitinated proteins and polymerizes through its PB1 domain to form aggregates that are targeted to autophagosomes for degradation. It was first shown to bind target associated ubiquitin and LC3 on the phagophore membrane, and is defined as a prototype autophagic receptor, which is found in cellular aggregates that links the autophagy process to polyubiquitinated proteins (Moscat, et al., 2007, Trends Biochem Sci, 32(2): 95-100). This adaptor protein polymerizes through its PB1 domain and consequently binds to the polyubiquitinated proteins and organelles such as depolarized mitochondria through its UBA domain, which facilitates protein aggregate formation.

PB1 is the oligomerization domain of the p62 protein, and binds to the PB1 domain of another p62 protein or other proteins which have PB1 domain. The UBA domain interacts with the ubiquitin-chain of the cargo to be delivered. This complex then interacts with LC3 through the LIR (LC3 interacting region) domain to join the autophagosomal machinery (Pankiv, et al., 2007, Journal of Biological Chemistry, 282(33): 24131-24145).

p62 also contains ZZ (zinc finger) and TB (TRAF6 binding) domains, which are involved in signaling via interaction with RIP and TRAF6 respectively (FIG. 2). RIP (ribosome inactivating protein) inhibits protein synthesis, and TRAF6 mediates the signaling of TNF receptor family (Moscat, et al., 2007, Trends Biochem Sci, 32(2): 95-100). In addition, p62 has been reported to interact with Keap1 to prevent Nrf2 degradation and promote Nrf2 mediated anti-oxidant response (Komatsu, et al., 2010, Nature Cell Biology, 12(3): 213-223). p62 has been identified as an integral part of mTORC1 complex while binding with Raptor and is necessary to mediate amino acid signaling for the activation of S6K1 and 4EBP1 (Duran, et al., 2011, Molecular Cell, 44(1): 134-146).

Autophagy has a dual role in cancer; it suppresses cancer initiation while enabling growth of aggressive cancers. As a stress response mechanism, autophagy prevents tissue damage that can promote cancer initiation and progression of early stage cancers. However, it is important for the integrity of tumor cells in later stages, and maintains mitochondrial metabolic function important for growth of aggressive cancers.

As in normal cells, tumor cells also require autophagy to survive metabolic stress, mainly in hypoxic regions (Degenhardt, et al., 2006, Cancer Cell, 10(1)151-64). p62 deficiency impairs Hras^{V12} induced tumorigenesis (Guo, et al., 2011, Genes & Development, 25(5): 460-470). Autophagy defects in mice lead to accumulation of polyubiqitinated p62 containing protein aggregates and unhealthy mitochondria (Komatsu, et al., 2005, The Journal of Cell Biology 169(3): 425-434; Mathew, et al., 2009, Cell, 137(6): 1062-1075; Yao, 2010, Genes & Cancer 1(7): 779-786; Takamura, et al., 2011, Genes & Development 25(8): 795-800).

Autophagy is a survival pathway utilized by tumor cells to survive hypoxic conditions in the tumor microenvironment where the vascularization is not enough. Therefore, autophagy inhibition may be an effective method to enhance cancer therapy.

The Ras family of small GTPases is among the group of proto-oncogenes most commonly mutated in human tumors; once activated, it stimulates downstream pathways important for transcription, cell cycle progression and cell survival (Downward, 2003, Nature reviews. Cancer 3(1): 1122) (FIG. 3).

Active Ras-expressing cells need functional mitochondria to support their high metabolic demand due to high proliferation rate. Under stress conditions, autophagy is the only mechanism for the cell to maintain healthy mitochondria. Ras activation upregulates basal autophagy and autophagy deficiency suppresses survival in oncogenic Ras induced tumor cells wider starvation conditions, and compromises tumor growth. Moreover, Ras activation in autophagy-defective cells causes specific reduction in citrate and isocitrate levels, and decreased flux through the TCA cycle (Guo, et al., 2011, Genes & Development, 25(5): 460-470).

p62 is activated in response to stress to conduct selective autophagic degradation. Failure to clear p62 and its cargo has been found to cause liver damage and promote tumorigenesis in allografts (Komatsu, et al., 2007, Cell, 131(6): 1149-1163; Mathew, et al., 2009, Cell, 137(6): 1062-1075. However, p62 is required for efficient tumorigenesis by Ras; p62 deficiency impairs Kras induced lung cancer and also inhibits tumorigenesis in allografts, which can be reverted by p62 reconstitution (Duran, et al., 2008, Cancer Cell, 13(4): 343-354; Guo, et al., 2011, Genes & Development, 25(5): 460-470). p62 is also involved in NF-KB signaling; and is required for Ras induced NF-KB activation (Duran, et al., 2008, Cancer Cell, 13(4); 343-354). In autophagy deficient cells, NF-KB activation is inhibited and this is dependent on p62 expression (Mathew, et al., 2009, Cell, 137(6): 1062-1075), indicating that p62 accumulation can also impair NF-KB activation and can cause tumorigenesis. Therefore, NF-KB may have a dual role in tumorigenesis through autophagy deficiency dependent p62 accumulation.

The p62 PB1 domain is a key region for oligomerization and aggregation. It functions to form polymers that are components of aggregated ubiquitinated proteins that are utilized by autophagy (Moscat, et al. 2006 Molecular Cell 23(5): 631-640; Nakamura, et al., 2010, Journal of Biological Chemistry, 285(3); 2077-2089). It can also oligomerize with other PB1 domains in different proteins, such as NBR1 (neighbor of BRCA1 gene; ovarian tumor antigen) and PKC (protein kinase C). Similar to p62, NBR1 has a LC3 interacting region and zinc finger domain that are common with p62. Through interactions with p62 via the PB1 domain, NBR1 is involved in autophagic degradation of ubiquitinated targets (Kirkin, et al. 2009, Journal of Biological Chemistry, 285(3): 2077-2089). PB1 but not LIR domain is determined to be important for p62 localization to the autophagosome formation site and p62 oligomerization through its PB1 domain is also required for the localization (Itakura and Mizushima 2011, The Journal of Cell Biology, 192(1): 17-27). Additionally, PB1 is important for autophagic clearance of non-ubiquitylated substrates, and the non-ubiquitylated substrate to be cleared does not have PB1 domain (Watanabe and Tanaka, 2011, Journal of Cell Science 124 (Pt 16): 2692-2701).

### BRIEF SUMMARY OF THE INVENTION

In certain aspects, the present invention relates to methods of inhibiting tumorigenesis in a cell comprising contacting a cell with a molecule that exhibits a function of the PB1 domain of p62.

In additional aspects, the invention relates to molecules that exhibit a function of the PB1 domain.

Further aspects relate to methods for making molecules that exhibit a function of the PB1 domain.

Additional aspects relate to methods of treatment for cancer comprising administration to a subject in need of treatment an effective amount of a composition comprising a molecule exhibiting a function of the PB1 domain.

Further aspects relate to cell lines that express a polypeptide having a function of the PB1 domain. Additional aspects relate to transgenic animals that express a polypeptide having a function of the PB1 domain.

Both the foregoing general description and the following detailed description are exemplary and explanatory, and are intended to provide further explanation of the invention as claimed. Other aspects, advantages, and novel features will be readily apparent to those skilled in the art from the following detailed description of the invention.

### BRIEF DESCRIPTION OF THE DRAWINGS

FIG. 1 demonstrates the main players in autophagosome formation and elongation.

FIGS. 2A-B demonstrate the domain organization of p62 and its role in autophagic regulation of cell signaling. A. Domain structure of p62 protein. B. p62 cargo receptor protein is responsible for trafficking damaged uibiquitinated proteins and organelles such as mitochondria.

FIG. 3 demonstrates downstream signaling of Ras. As Ras is in GTP-bound state and active, it will interact with several families of effector proteins, resulting in stimulation of their catalytic activity.

FIG. 4 provides a p62 oligomerization model. Under normal conditions, p62 delivers its cargo proteins/organelles to the autophagosome for degradation. When there is excess amount of PB1, it binds to p62 which may inhibit its function.

FIG. 5A-B demonstrates the effect of PB1 as a dominant negative, domain in Ras induced tumorigenesis. A. PB1 expression inhibits Ras induced tumorigenesis. GFP and GFP-PB1 transfected W2-Ras cell lines were subcutaneously injected into immunocompromised mice and tumor sizes were monitored. B. PB1 domain is sufficient to reconstitute p62's tumor promoting effect. Ras-expressing p62 deficient cells transfected with GFP and GFP-PB1 to observe the effect of PB1 domain in a p62-deficient background. Error bars represent standard errors. P<0.05 (t-test).

FIG. 6 illustrates that PB1 expression attenuated tumor growth in Ras loop mutants Tumorigenesis. HRasV12G37, H-RasV12C40 and W2-RafCAAX illustrates the Ras loop mutants for RAL-GDS, PI3K/AKT and Raft representatively. Transfected cell lines were subcutaneously injected to the immonucompromised mice and tumor sizes were monitored. Error bars represent standard errors. P<0.05 (t-test).

FIGS. 7A-B demonstrate that PB1 expression in H1299 cells inhibited tumor growth in immunocompromised mice.

FIG. 8 shows immunohistochemistry for p62 in tumors from mice injected. with GFP and GFP-PB1 transfected W2-Ras cell lines.

FIG. 9 demonstrates that in vivo induction of PB1 expression causes p62 accumulation. PB1 expression was detected by RT-PCR and western blot. p62 antibody is used to detect PB1 and p62 levels. B-actin was used as loading control.

FIG. 10 shows the results of immunohistochemistry analysis demonstrating that in vivo induction of PB1 expression causes p62 accumulation in LSL-K-ras^{G12D} background.

FIGS. 11A-C demonstrate that PB1 expression inhibits K-ras induced lung tumor burden in mice. A. Gross pathology of lung lobes 4 weeks after doxycycline treatment. B. Wet weight measurements of mouse lungs shown in FIG. 11A. C. Paraffin embedded H&E stained slides of mouse lung sections shows the difference in tumor burden.

FIG. 12 illustrates structure based sequential alignment of PB1 domain of p62 protein containing OPCA motif and Lys residue on the opposite sides of each other. Acidic residues are indicated with red circles (and are within the OPCA motif) and basic residues are indicated with blue circles (by using RCSB Protein Data Bank).

FIG. 13A-B illustrates a model for PB1 self-interaction. A. Acidic and basic amino acids on either sides of PB1. B. Electrostatic surface potential of PB1 domain (by using PyMOL).

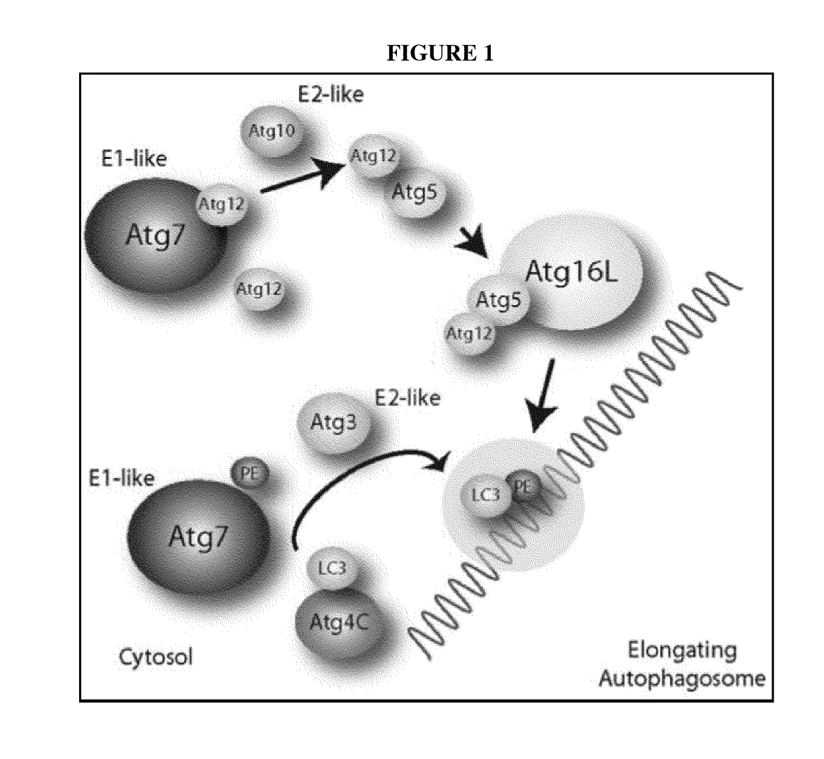

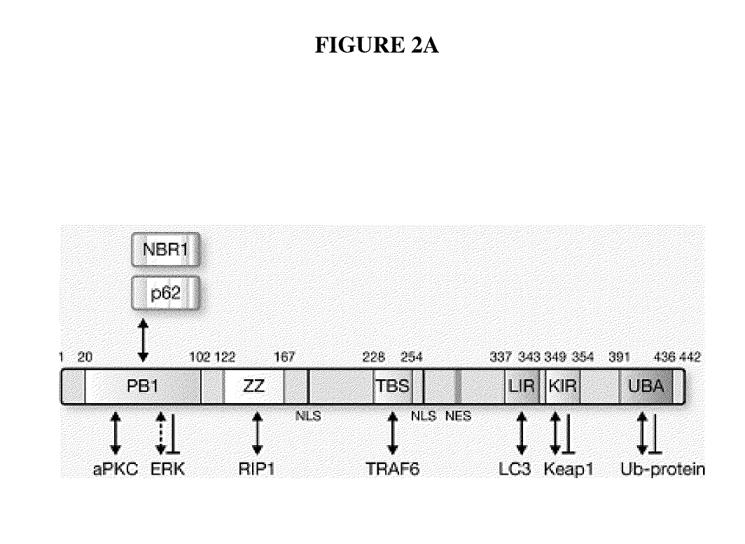

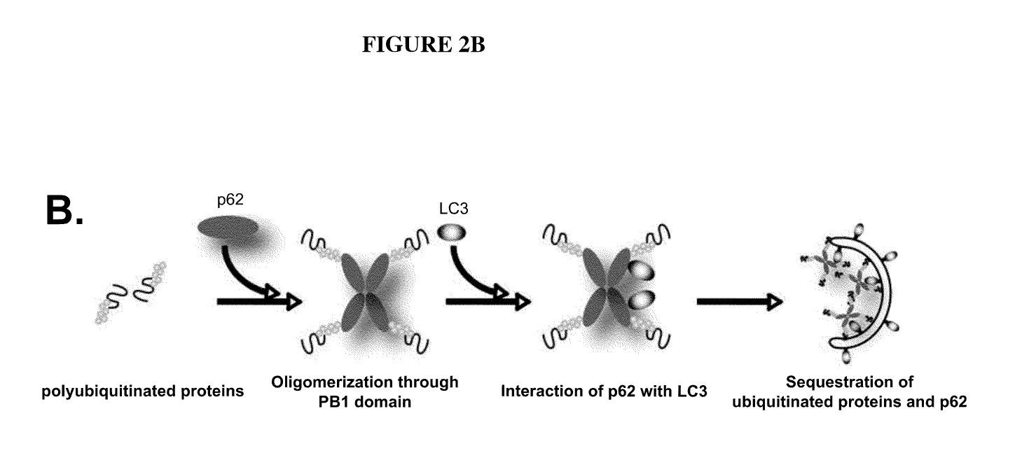

### DETAILED DESCRIPTION OF THE INVENTION

In certain aspects, the invention relates to compositions, methods, and kits using the functionality of the PB1 domain of p62 as a dominant negative for p62. In certain embodiments, a molecule having a function of the PB1 domain may be used. In certain embodiments, a PB1 domain or PB1 mimetic functions by binding the p62 PB1 domain and inhibiting self-oligomerization of p62. In certain embodiments, a PB1 domain or PB1 mimetic may be used as an anti-cancer agent.

Certain embodiments relate to a method of inhibiting tumorigenesis in a cell comprising contacting the cell with a molecule that exhibits a function of the PB1 domain of p62. In certain embodiments, the tumorigenesis may be induced by a expression of a Ras protein (Ras-induced). In certain embodiments, a function of the PB1 domain comprises binding to the p62 PB1 domain. In certain embodiments, contacting the cell with the molecule may lead to accumulation of p62 in the cell. In some embodiments, the molecule may be a polypeptide. In some embodiments, the molecule may be other than a polypeptide, such as, without limitation, a small molecule that mimics a function of the PB1 domain.

In certain aspects, the invention relates to a molecule that mimics a function of the PB1 domain of p62. In certain aspects, the function of the PB1 domain that is exhibited by the molecule comprises binding to the p62 PB1 domain. In certain aspects, the PB1 mimetic molecule may be a polypeptide. Preferably, the polypeptide may be isolated, purified, and/or synthetic. Additional embodiments relate to an isolated polynucleotide comprising, a nucleotide sequence encoding the polypeptide. In certain embodiments, the polynucleotide may comprise a nucleotide sequence encoding the polypeptide operatively linked to at least one expression control sequence effective for expression of the polypeptide. In certain aspects, the molecule may be other than a polypeptide, such as, without limitation, a small molecule that mimics a function of the PB1 domain.

Further embodiments relate to a pharmaceutical composition comprising a molecule that exhibits a function of the PB1 domain of p62. As described herein, such a molecule may include, without limitation, an isolated polypeptide comprising the PB1 domain, an isolated polypeptide comprising a variant thereof having a PB1 function, or other PB1 mimetic. As described herein, a PB1 mimetic may include, without limitation, a polypeptide or a small molecule mimetic. The use of isolated polynucleotides encoding the relevant polypeptides is also contemplated.

In additional aspects, the invention relates to methods of identifying PB1 mimetics. In certain embodiments, the invention relates to methods of identifying the smallest effective sequence of the p62-PB1 domain capable of exhibiting a function of the p62 PB1 domain. In certain embodiments, the function comprises binding to the p62 PB1 domain. In certain embodiments, the PB1 mimetic inhibits p62 self-oligomerization. In certain embodiments, the PB1 mimetic has anti-tumor activity.

Additional embodiments relate to a method of making a molecule that exhibits a function of the PB1 domain of p62. In certain embodiments, the method may comprise the synthesis of a peptide having a function of the PB1 domain.

In certain embodiments, methods of assessing the role of PB1 in a Ras-induced lung carcinoma mouse model are provided. Further aspects include the investigation of the mechanism by which PB1 inhibits has induced tumorigenesis through identification of PB1-interacting proteins.

Additional aspects include methods for the identification of the shortest effective amino acid sequence of PB1 in inhibiting Ras-induced tumorigenesis. Further aspects include the design of molecules to mimic PB1 function. In certain embodiments, this molecule may be a polypeptide.

Another embodiment relates to a kit comprising a molecule that exhibits a function of the PB1 domain of p62 and at least one container.

Additional aspects relate to a cell line comprising a cell that expresses a polypeptide haying a function of the PB1 domain. Preferably, the cell stably expresses a polypeptide having a function of the PB1 domain. In certain aspects, the cell line comprises a cell that further expresses an oncogenic protein. Preferably, the oncogenic protein is a has protein.

Further embodiments relate to a non-human transgenic animal that expresses a polypeptide having a function of the PB1domain. In certain embodiments, the polypeptide having a function of the PB1 domain is conditionally expressed.

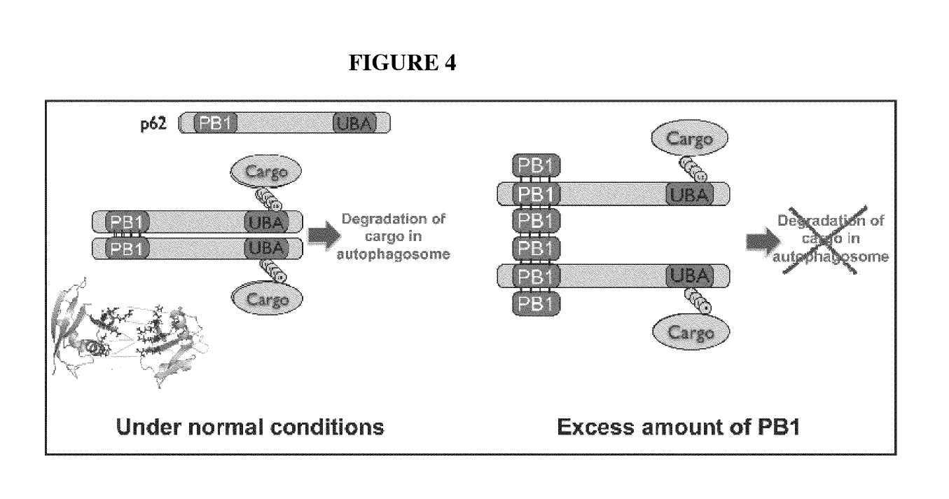

While not intending to be bound by any theory of operation, manipulating the p62 oligomerization step may block the machinery required to recycle autophagic substrates, which are needed for energetic demand and organelle function. p62 oligomerization is important to deliver polyubiquitinated proteins to the autophagosome machinery for degradation and recycling. The PB1 domain is important for self-oligomerization of p62, which is required for trafficking of unfolded proteins in autophagy. Preventing p62 self-oligomerization by expression of the p62 PB1 domain inhibits p62 degradation and causes accumulation of p62 aggregates (FIG. 4).

Ras-expressing cancer cells require autophagy to survive metabolic stress and to grow as tumors. Without intending to be bound by any theory of operation, inhibition of autophagy with PB1 in autophagy “addicted” tumor cells may be used to improve the efficacy of cancer therapeutics. The group of protooncogenes most commonly mutated in human tumors is the Ras family, which is involved in cellular signal transduction; the signals derived from Ras pathway result in cell growth and division. A mutation in a Ras protein causing deregulation may induce oncogenesis. Mutations in Ras and downstream kinases are common in cancer; however, there are no direct inhibitors identified for clinical use. An alternative approach to inhibiting Ras-driven tumors is to target the downstream metabolic events, such as, for example, modulating an alternative mechanism such as autophagy, that Ras-induced tumor cells use to survive.

While not intending to be bound by any theory of operation, preventing self-oligomerization appears to inhibit p62 function in the cargo delivery process of autophagy in Ras-induced tumorigenesis. In certain embodiments, the PB1 domain may be used as a dominant negative for p62, by binding its p62-PB1 domain and inhibiting its self-oligomerization.

In certain embodiments of the invention, methods are used to identify the role of PB1 in Ras-induced tumorigenesis. In certain embodiments, transgenic mouse models are used. In certain embodiments, in vitro culture of human cancer cell lines are used. In certain embodiments, methods are employed to define the smallest effective amino acid sequence that mimics a function of the PB1 domain. In certain embodiments, PB1 -interacting molecules are exploited for cancer therapy.

Another aspect of the invention provides a method of treatment for cancer comprising administration to a subject in need of treatment an effective amount of a composition that comprises, or enables the in vivo production of, a molecule that exhibits a function of the PB1 domain.

In certain embodiments, compositions are formulated with pharmaceutically acceptable diluents, adjuvants, excipients, or carriers. The phrase “pharmaceutically or pharmacologically acceptable” refers to molecular entities and compositions that do not produce adverse, allergic, or other untoward reactions when administered to an animal or a human, e.g., orally, topically, transdermally, parenterally, by inhalation spray, vaginally, rectally, or by intracranial injection. The term parenteral as used herein includes subcutaneous injections, intravenous, intramuscular, intracistemal injection, or infusion techniques. Administration by intravenous, intradermal, intramusclar, intramammary, intraperitoneal, intrathecal, retrobulbar, intrapulmonary injection and/or surgical implantation at a particular site is contemplated as well. Generally, this will also entail preparing compositions that are essentially free of pyrogens, as well as other impurities that could be harmful to humans or animals. The term “pharmaceutically acceptable carrier” includes any and all solvents, dispersion media, coatings, antibacterial and antifungal agents, isotonic and absorption delaying agents, liposomes, capsids, nanocapsules, microcapsules and the like. The use of such media and agents for pharmaceutically active substances is well known in the art.

In certain embodiments, the present invention provides a method of treating a subject comprising administration of a composition. As used herein, the term “subject” is used to mean an animal, preferably a mammal, including a human. The terms “patient” and “subject” may be used interchangeably.

In certain embodiments, the therapeutic agents of the present invention may be used alone or in combination with other cancer therapies including, but not limited to, chemotherapy, radiation therapy, immunotherapy and gene therapy.

In another embodiment, the method of treatment further comprises the administration of a second therapeutic agent. In a preferred embodiment, the second therapeutic agent is an anticancer agent. In such an embodiment, the second agent may be administered before, after, or concurrently with the compositions of the present invention.

Techniques for formulation and administration of the therapeutic compositions of the instant application may be found in “Remington's Pharmaceutical Sciences,” Mack Publishing Co., Easton, Pa., latest edition. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

The therapeutic compositions may be administered by any route that delivers an effective dosage to the desired site of action, with acceptable (preferably minimal) side-effects.

An effective amount of the compositions to be employed therapeutically will depend, for example, upon the therapeutic objectives, the route of administration, and the condition of the patient. Accordingly, it may be necessary for the therapist to titer the dosage and modify the route of administration as required to obtain the optimal therapeutic effect.

Therapeutic dosing is achieved by monitoring therapeutic benefit and monitoring to avoid side-effects. Preferred dosage provides a maximum localized therapeutic benefit with minimum local or systemic side-effects. Suitable human dosage ranges for the polynucleotides or polypeptides can be extrapolated from these dosages or from similar studies in appropriate animal models. Dosages can then be adjusted as necessary by the clinician to provide maximal therapeutic benefit for human subjects.

Autophagy is found to be required for the optimal growth of tumor cells transformed with oncogenic H-RasV12 and K-RasV12. PB1 is not a p62 specific domain; i.e., there are other proteins have PB1 domains and interact with p62; therefore it should be considered that other interacting proteins such as, for example, PKC and NBR1, via their PB1 domain, may interfere with the results. Additionally, NBR1 has an LC3 interacting region and a zinc finger domain that are common with p62. Through its interactions with p62 via the PB1 domain; NBR1 is involved in autophagic degradation of ubiquitinated targets, hence it may be useful to assess NBR1 deficient background as well. There may be variability of the results since the expression level of PB1 is inducible, but not controllable. To address this, different mouse lines may be used bearing a PB1 construct. Three mouse lines have been tested; each have similar mRNA levels, but varying protein levels. PB1 expression does not appear to induce toxicity in mice.

The results provide herein demonstrate that preventing p62 self-oligomerization by expression of the p62 PB1 domain inhibits p62 degradation and causes p62 aggregates accumulation that cannot be degraded through autophagy. It is also demonstrated that PB1 expression inhibits tumor growth in Ras allografts and LSL-Kras^{G12D} transgenic mice.

The following examples serve to further illustrate the present invention.

### EXAMPLE 1

### PB1 Expression Inhibits Ras Induced Tumorigenesis

Ras expressing cancer cells require autophagy to survive metabolic stress and to grow as tumors. Isogenic immortalized baby mouse kidney epithelial (iBMK) cell lines, which are autophagy competent (atg5+/+) and deficient (atg5−/−), were transfected with a strong cell growth-promoting activated oncogene H-ras^{V12}. According to the clonogenic survival assay and tumor formation induction analysis in immunocompromised mice, deficiency in the essential autophagy gene atg5 impairs both survival under starvation and tumorigenesis, which indicates a functional requirement for autophagy in Ras-driven oncogenesis. Knowing that p62 is an important cargo receptor in autophagy and responsible for cargo sequestering, to the autophagosome, loss of p62 also impaired H-ras^{V12} induced tumorigenesis. (Guo, J. Y., et al. 2011, Genes & Development, 25(5): 460-470).

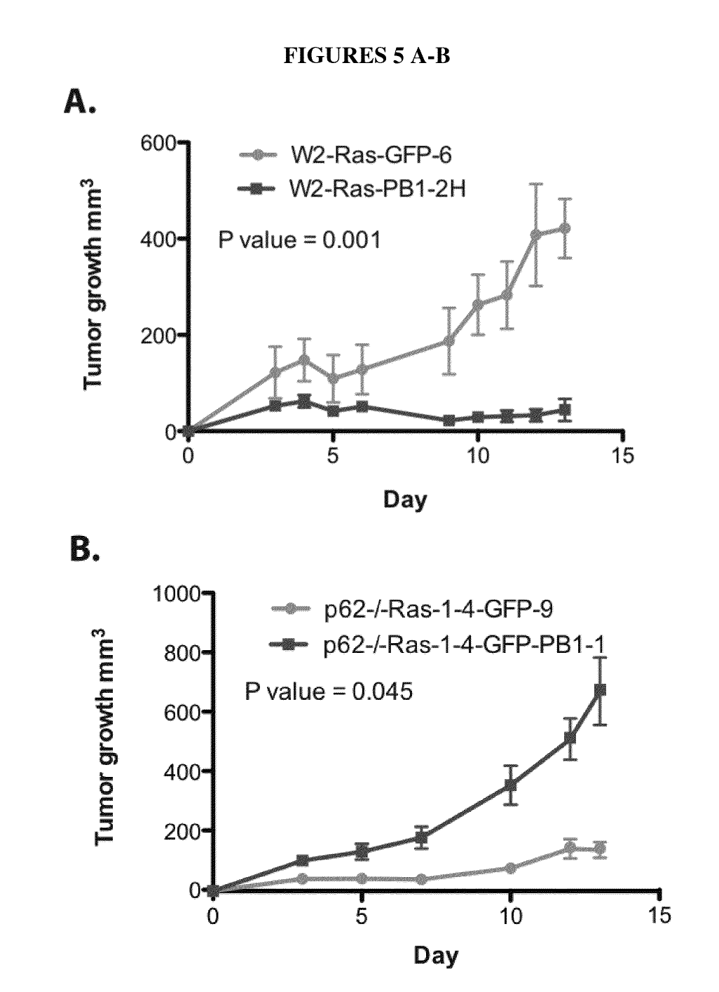

After observing that p62 is required for Ras induced tumorigenests, it was determined whether the PB1 domain of p62 can act as a dominant negative to inhibit p62 function and prevent Ras activation induced tumorigenesis. The GFP-tagged PB1 domain of p62 protein was stably expressed in iBMK W2 cells (Degenhardt, et al, 2002, Cancer Cell 2(3): 193-203) with Hras^{V12}. Subsequently, W2-Hras^{V12}-PB1 cells were subcutaneously injected into immunocompromised mice and tumor growth was monitored for two weeks. The GFP clone was used as a control to compare tumor size in the normal condition. PB1 domain expression inhibited Hras^{V12} induced tumorigenesis and by itself it was sufficient to reconstitute p62 effect in p62-deficient cell induced tumorigenesis, which indicates a dominant negative effect of the PB1 domain (FIG. 5). Error bars represent standard errors.

### EXAMPLE 2

### PB1 Expression Attenuates Tumor Growth in Ras Loop Mutant Tumorigenesis

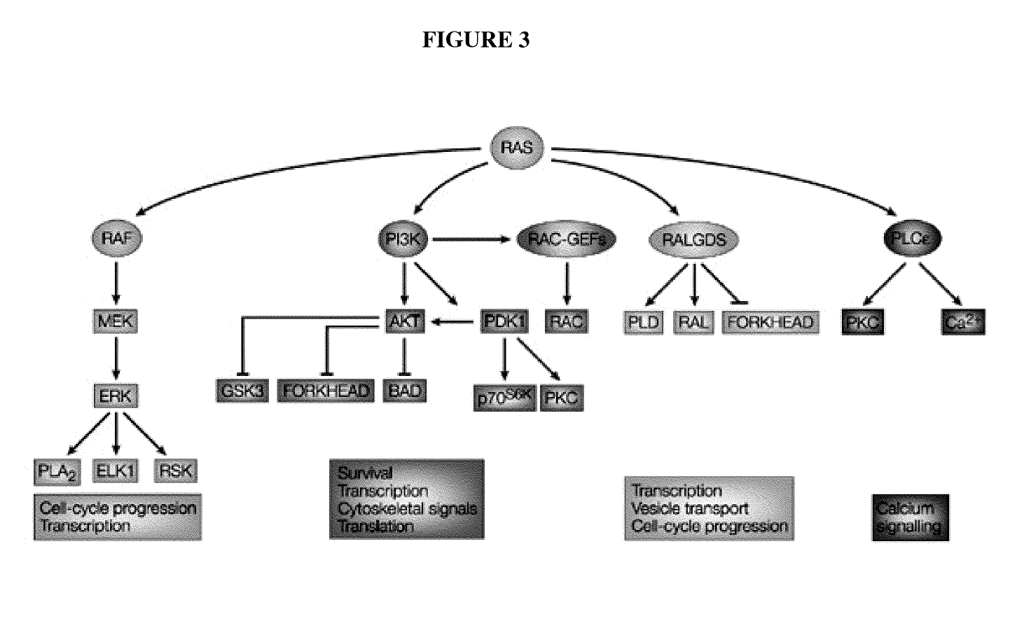

As shown in FIG. 3, Ras activation triggers downstream pathways as RAF, PI3K, RALGDS and. PLCε. To investigate the molecular mechanism of PB1 domain on Ras induced tumorigenesis, GFP-PB1 stably expressed in Ras effector loop mutants were tested in allografts. Transfected cell lines were subcutaneously injected into immunocompromised mice and tumor sizes were monitored.

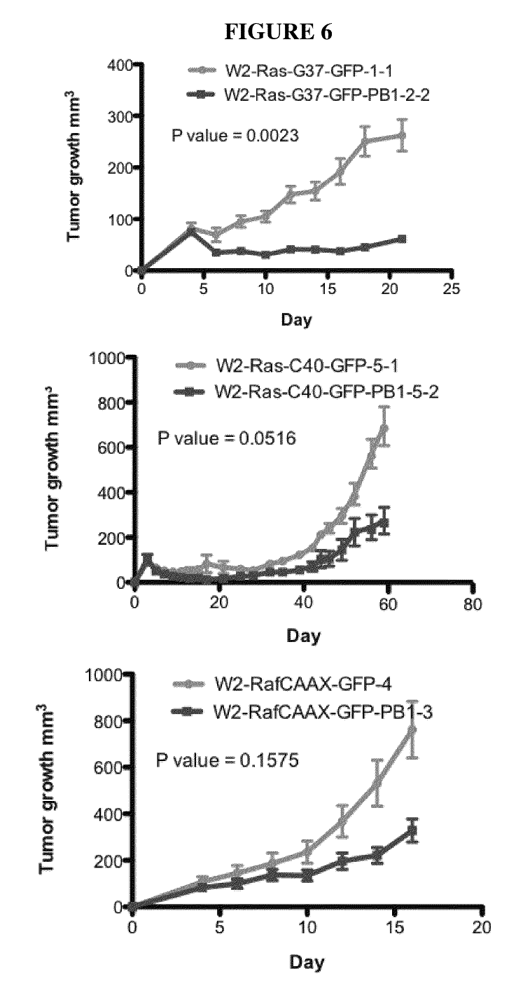

HrasV12G37 is a Ras effector domain mutant that specifically activates RAL-GDS Pathway; HrasV12C40 preferentially activates the PI3K/AKT pathway; and W2-RafCAAX is constitutively localized to the membrane, which induces constitutive Raf pathway activation. Individual expression of GFP-PB1 in W2-HrasV12G37, W2-HrasV12C40, or W2-RafCAAX iBMK cells profoundly reduced Ras loop mutant induced tumor growth in immunocompromised mice compared to a GFP vector control (FIG. 6). Error bars represent standard errors.

### EXAMPLE 3

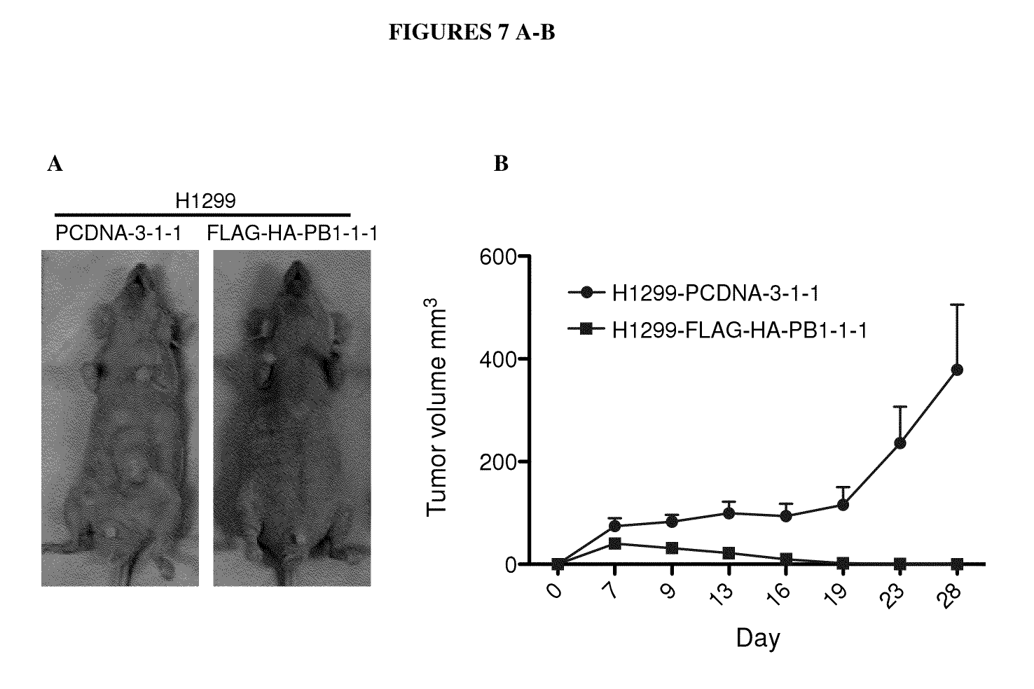

### PB1 expression in H1299 cells, human non-small lung cancer cells which bear a Ras mutation, inhibited tumor growth in immunocompromised mice. (FIG. 7).

### EXAMPLE 4

### PB1 Expression Causes p62 Accumulation in Ras Induced Tumorigenesis

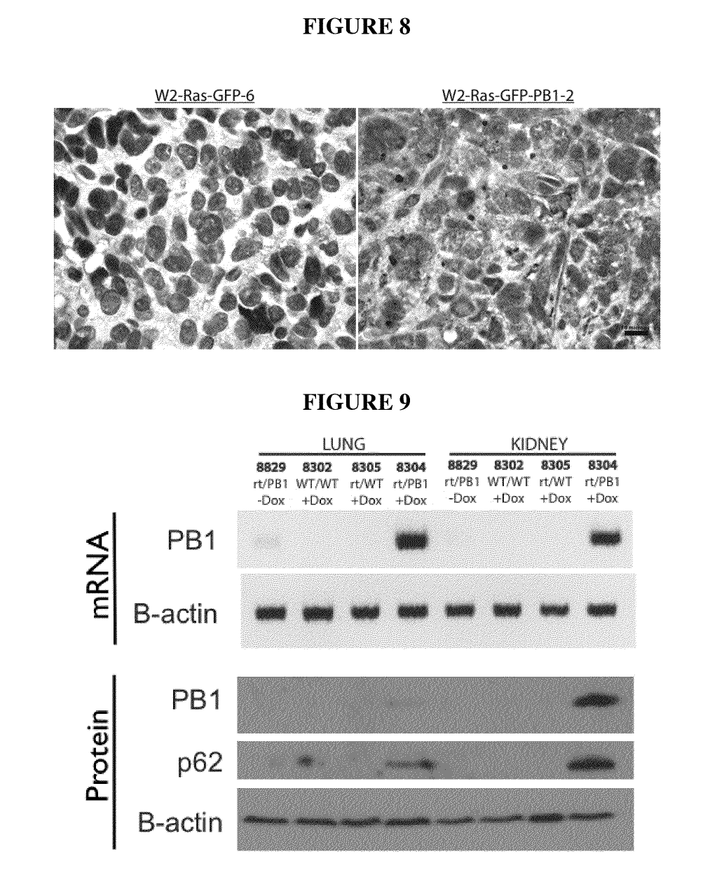

Immunohistochemistry was conducted for p62 in tumors from mice injected with GFP and GFP-PB1 transfected W2-Ras cell lines (W2-Hras^{V12}-GFP and W2-Hras^{V12}-GFP-PB1 cells induced tumor tissues). High levels of p62 accumulation were detected in W2-Ras-GFP-PB1 induced tumor samples as a consequence of PB1 expression, which indicates that abnormal p62 aggregation cannot be degraded through autophagy (FIG. 8).

### EXAMPLE 5

### In Vivo Induction of PB1 Expression Leads to p62 Accumulation

To examine the therapeutic potential of inhibiting endogenous p62 function in cancer, a mouse model was generated in which PB1 may be conditionally expressed and its effect on p62 function in vivo may be determined. PB1 was conditionally expressed under the CMV promoter, to induce its expression in multiple tissues. The PB1 coding sequence was placed downstream of a tetracycline-response promoter element (TRE). Mice having TRE-PB1 transgene were crossed to B-actin-rtTA, to conditionally express PB1 dependent on doxycycline treatment administered in drinking water. RT-PCR and western blot results confirmed that PB1 expression was detectable in multiple tissues including, lung and kidney in TRE-PB1; B-actin-rtTA mice on doxycycline treatment. In addition, p62 accumulation was correlated with PB1 expression and no PB1 expression was detected in TRE-PB1 mice or wild type mice (FIG. 9). p62 antibody was used to detect PB1 and p62 levels. B-actin was used as a loading control.

### EXAMPLE 6

### Assessment of the Role of PB1 in Ras Induced Lung Carcinoma Mouse Model

To test the hypothesis that inhibition of p62 function can inhibit Ras driven spontaneous cancer, a model was generated with TRE-PB1; B-actin-rtTA;LSL-K-ras^{G12D} compound mutant mice, in which PB1 expression may function as a dominant negative to inhibit p62 function. Therefore, PB1 may be conditionally expressed under the control of tetracycline response element in a doxycycline-dependent manner.

B-actin promoter was fused to rtTA transactivator, which is a positive doxycycline dependent reverse transcriptase transactivator, it binds to tetracycline response elements (TRE) in the presence of doxycycline and causes transcriptional activation of genes downstream of TRE. As the expression of PB1 was confirmed in mRNA and protein levels (FIG. 9), TRE-PB1;B-actin-rtTA mice were crossed to LSL-K-ras^{G12D}. G12D denotes a point mutation for constitutive activation of Kras that is flanked by loxP sites. Upon inhalation of adenovirus expressing cre recombinase (Ad-Cre), the oncogenic K-ras^{G12D} allele is activated in a background of conditional PB1 expression, and the compound mutant mice were tested for the consequences to tumorigenesis.

p62 accumulation has been detected in lung, in TRE-PB1;B-actin-rtTA; LSL-K-ras^{G12D} mice, by both western blot analysis and immunohistochemistry; but not in the control group expressing PB1 and rtTA transactivator expression alone.

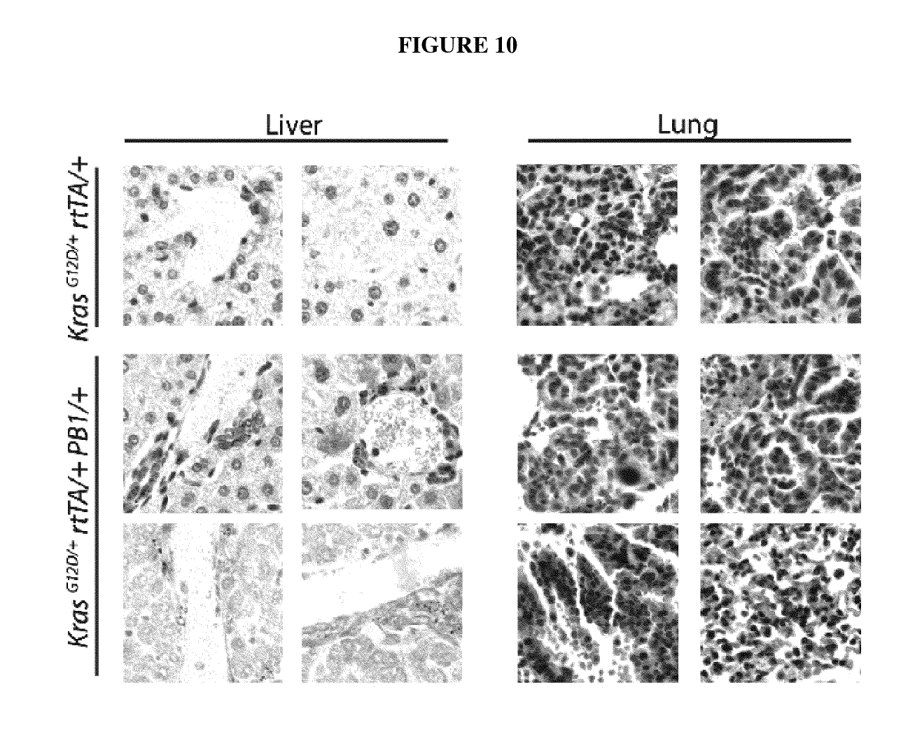

Immunohistochemistry analysis demonstrates that in vivo induction of PB1 expression causes p62 accumulation in the LSL-K-ras^{G12D} background (FIG. 10). Mice were treated with doxycycline at 2 weeks post-Cre infection and dissected 5 weeks after treatment.

### EXAMPLE 7

### PB1 Expression Inhibits K-ras Induced Lung Tumor Burden

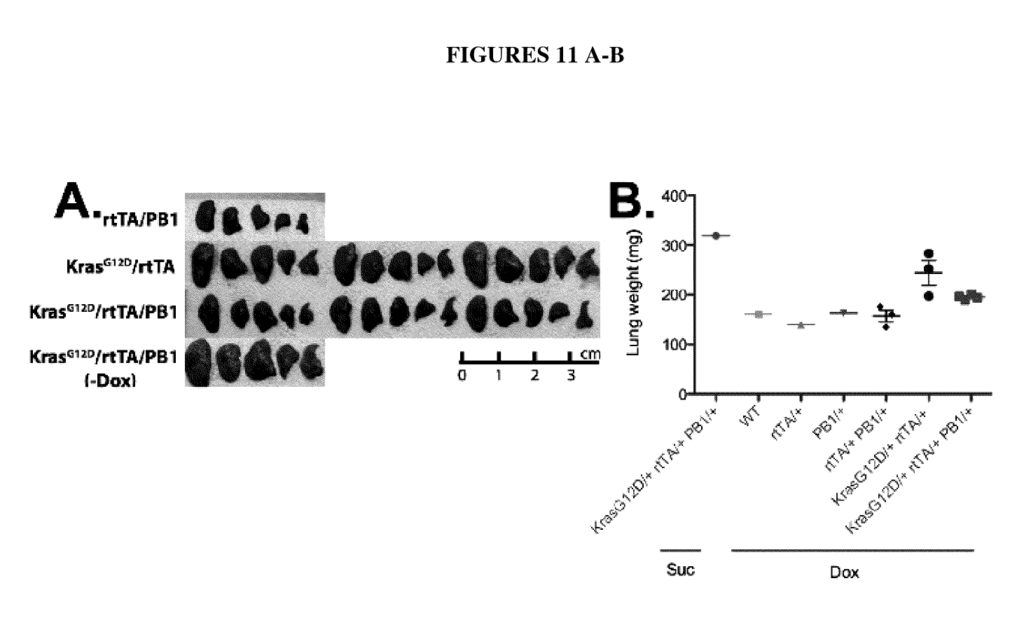

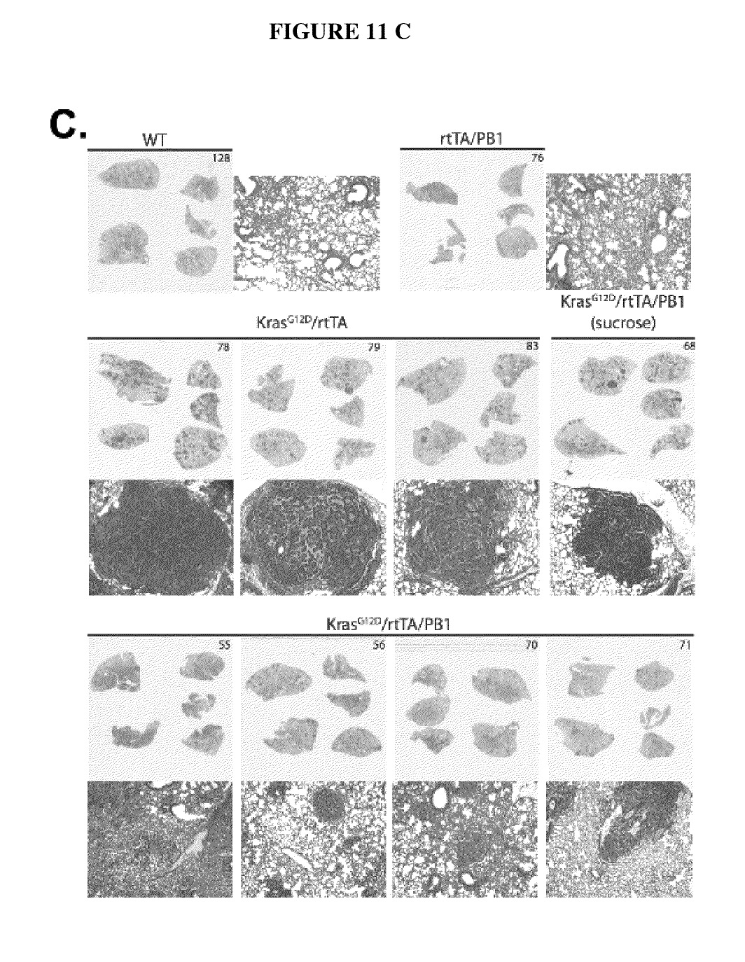

FIG. 11A demonstrates the gross pathology of lung lobes 4 weeks after doxycycline treatment. Lungs are smaller in PB1 expressing LSL-Kras^{G12D} transgenic mice. Mice were infected with adenoviral Cre at 6-9 weeks of age, and were treated with doxycyline at 10 weeks post-Cre administration. Doxycycline concentration was 2 mg/ml and was dissolved in 5% sucrose. FIG. 11B demonstrates the wet weight measurements of mouse lungs shown in FIG. 11A. Paraffin embedded H&E stained slides of mouse lung sections shows the difference in tumor burden. See FIG. 11C. These results demonstrate that PB1 expression in Kras/rtTA/PB1 mice causes a decrease in adenoma size.

### EXAMPLE 8

For K-ras induced spontaneous tumors, hyperplasia occurs at 2-3 weeks (grade 1 lesions), early adenomas at 6-12 weeks (grade 2 lesions), and adenocarcinomas at 16 weeks (grade 3 lesions), with lethality by 7 months without invasion or metastasis (DuPage, et al, 2009, Nature Protocols, 4(7): 1064-1072). Upon maintenance of early adenomas around 6-12 weeks (grade 2 lesions), treatment is started with doxycycline (which is a member of tetracycline antibiotics group). Doxycycline treatment is dosed at 6 weeks and 10 weeks post-cre infection and the effect of PB1 expression on tumor initiation and maintenance is monitored. The effect of PB1 expression on late stage tumor growth through doxycycline treatment at 14 weeks post-cre infection is also assessed. The extent of PB1 expression on tumor growth is assessed by immunohistochemistry of certain autophagy, cell cycle, MAPK signaling and immune response marker proteins. Additionally, viability, tumor stage, metastasis, metabolite flux are determined and tumor volumes are monitored by micro-computed tomography.

### EXAMPLE 9

### Identification of the Shortest Effective Sequence of PB1 in Ras Induced Tumorigenesis and Design of a Polypeptide to Mimic PB1 Function

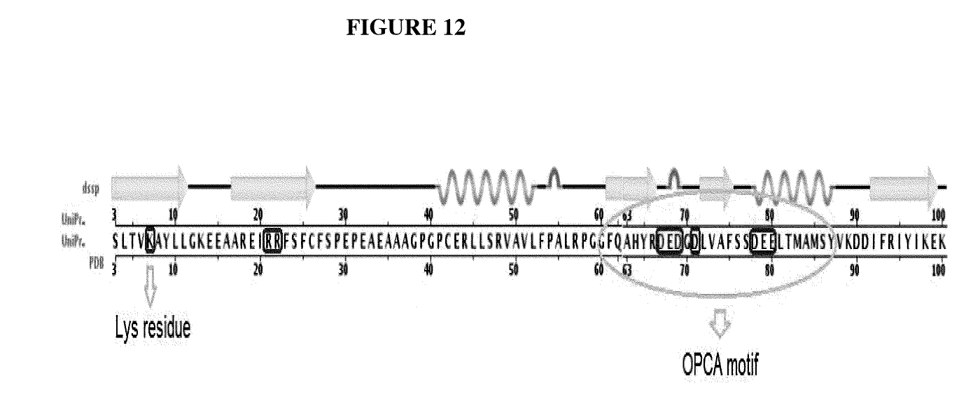

To design an oligopeptide that mimics the PB1 domain's function, it is determined whether the entire domain is required or if a specific sequence within the domain is sufficient. NMR structure of p62 PB1 domain has been determined (Saio, et al., 2009, Journal of Biomolecular NMR 45(3): 335-341). The PB1 domain is classified into three types according to the motifs it has; type I, type II and type I/II. Type I has OPCA motif, which is a 28 amino acid region, highly acidic and hydrophobic; whereas type II has a conserved lysine (Lys) residue on exactly opposite site of OPCA motif. Type I/II contains both of these regions and hence is appropriate for self-interaction in a front-back topology (Hirano, et al., 2004, The Journal of Biological Chemistry 279(30): 31883-31890). As indicated in FIG. 9, the p62 PB1 domain is a type I/II PB1 domain bearing both an OPCA motif and Lys residue (FIG. 12).

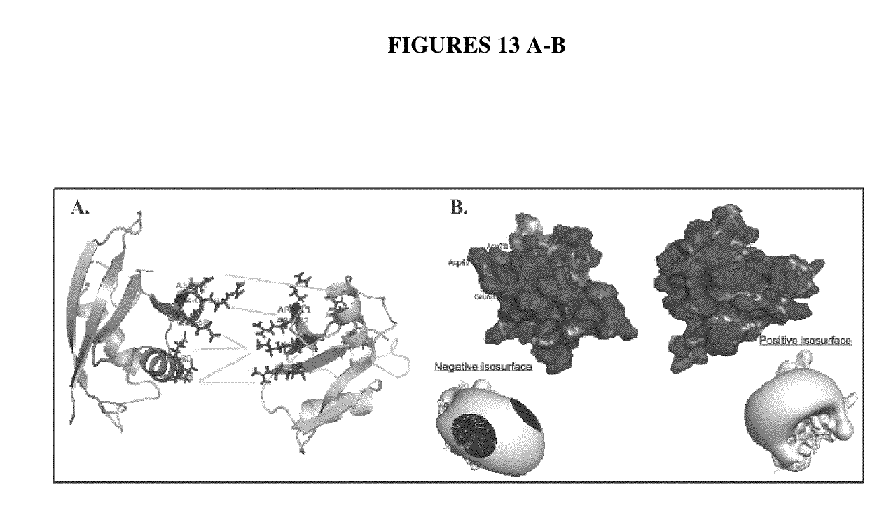

The PB1 domain of p62 protein, being a type I/II PB1, has basic amino acids—including Lys7—close to the N-terminus of the domain; and acidic amino acids in the OPCA motif, which is close to the C-terminus. Without intending to be bound by any theory of operation, these observations may explain front-back interaction. In FIG. 13A an oligomerization structure according to these amino acids is modeled. Acidic and basic surfaces are highly compartmentalized on either side of the domain, which indicates electrostatic interactions likely play an important role in self-interaction. Electrostatic surface potentials are shown in FIG. 13B using APBS (Adaptive Poisson-Boltzmann Solver) program of PyMOL. As expected, the acidic and basic surfaces form extreme electrostatic surfaces making the structure available for an electrostatic interaction; which may represent a model for front-back topology interaction (FIG. 13).

To determine the shortest effective sequence of the PB1 domain, several sequential deletion constructs, which specifically target the OPCA motif where the acidic and basic residues reside, are generated by PCR and tested. These constructs are stably expressed in iBMK and human cancer cells with Ras mutations. Starvation assays are conducted to test the consequence of p62 inhibition on cell survival under starvation.

Dominant negative function is examined by injecting those cells in immunocompromised mice and monitoring rumor growth. Once the shortest effective PB1 domain is identified, genetically modified mice are generated as described herein and function is confirmed. One or more peptides are synthesized. The effect of such peptides are examined on oncogenic Ras-induced lung tumors in vivo.

### EXAMPLE 10

### Investigating the Mechanism by Which PB1 Inhibits Ras-Induced Tumorigenesis Through Identification of PB1 Interacting Proteins

As the region of PB1 inhibiting Ras driven tumor is identified, the mechanism by which it inhibits Ras induced tumorigenesis is investigated. To analyze protein-protein interactions of PB1 domain, tagged PB1 constructs are used for tandem affinity purification (TAP). Recently, it has been shown that p62 can bind to non-ubiquitylated substrates through its PB1 domain for their autophagic clearance (Watanabe and Tanaka, 2011, Journal of Cell Science 124(Pt 16): 2692-2701). These methods are used to identify specific interactions required for autophagy and Ras-induced tumorigenesis.

PB1 constructs are generated and tagged with FLAG and HA. The tagged PB1 construct is expressed in IBMK cells. Protein-protein interactions are analyzed by tandem affinity purification. As an advantage of the system, the interacting proteins are determined quantitatively in vivo without prior knowledge of complex composition. For this purpose, first an anti-FLAG resin is directly added to the lysate and then the resin is transferred to a spin column, then it is eluted with FLAG peptide. The same steps are conducted with an anti-HA affinity resin as well. Then the eluted interacting proteins are identified by mass spectrometry. After two affinity purifications the chance for contaminants to be retained in the elute is low. As the interacting proteins are identified, knock-down of the proteins using shRNA are used to confirm the mechanism in which PB1 is involved in blocking p62 function and as a result inhibiting tumorigenesis.

Knock-down experiments are repeated in p62 deficient IBMK cells to determine whether the interaction between PB1 and the target proteins also involves p62 i.e. to identify if there is a direct or indirect interaction.

The terms and expressions which have been employed are used as terms of descriptions and not of limitation, and there is no intention that in the use of such terms and expressions of excluding an equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

In addition, where features or aspects of the invention are described in terms of Markush group or other grouping of alternatives, those skilled in the art will recognize that the invention is also thereby described in terms of any individual member or subgroup of members of the Markush group or other group.

All references cited herein are incorporated herein by reference in their entireties.

What is claimed is:

1. A method of inhibiting tumorigenesis in a cell comprising contacting the cell with a molecule that exhibits a function of the PB1 domain of p62.

2. The method of claim 1 wherein the tumorigenesis is Ras-induced.

3. The method of claim 1 wherein the function comprises binding to the p62 PB1 domain.

4. The method of claim 1 wherein contacting the cell with the molecule causes inhibition of self-oligomerization of p62.

5. The method of claim 1 wherein the molecule is a polypeptide.

6. A molecule that exhibits a function of the PB1 domain of p62.

7. The molecule of claim 6 wherein the function comprises binding to the p62 PB1 domain.

8. The molecule of claim 6 wherein the molecule is a polypeptide.

9. An isolated polynucleotide comprising a nucleotide sequence encoding the polypeptide of claim 8.

10. The polynucleotide of claim 9, wherein the nucleotide sequence encoding the polypeptide is operatively linked to at least one expression control sequence effective for expression of the polypeptide.

11. A pharmaceutical composition comprising a molecule that exhibits a function of the PB1 domain of p62.

12. The composition of claim 11 wherein the molecule is a polypeptide.

13. A method of treatment for cancer comprising administration to a subject in need of treatment an effective amount of a composition comprising a molecule exhibiting a function of the PB1 domain of p62.

14. the method of claim 13 wherein the the function comprises binding to the p62 PB1 domain.

15. A method of making a molecule that exhibits a function of the PB1 domain of p62 comprising a step of producing a polypeptide having said function.

16. The method of claim 15 wherein the function comprises binding to the p62 PB1 domain.

17. A kit comprising a molecule that exhibits a function of the PB1 domain of p62 and at least one container.

18. A cell line comprising a cell that stably expresses a polypeptide having a function of the PB1 domain.

19. The cell line of claim 18 wherein the cell further expresses an oncogenic Ras protein.

20. A non-human transgenic animal that conditionally expresses a polypeptide having a function of the PB1 domain.
